(19) United States
(12) Patent Application Publication
(10) Pub. No.: US 2008/0075674 A1
(43) Pub. Date: Mar. 27, 2008

(54) Cough Suppressor

(76) Inventor: Brian D. DuPratt, Olmsted Falls, OH (US)

Correspondence Address: HARSHAW RESEARCH, INC., 210 W. TECUMSEH STREET, OTTAWA, KS, 66067, US

(21) Appl. No.: 11/534,784

(22) Filed: Sep. 25, 2006

(51) Int. Cl. A61K 9/68

(52) U.S. Cl. 424/48; 424/440

(57) ABSTRACT

A cough suppressor according to the present invention includes a chewable inner layer having a predetermined flavor. Preferably, the inner layer is chewing gum. The cough suppressor also includes an active layer situated outside the inner layer and that includes a cough suppressant. In addition, a buffer layer completely separates the inner layer from the active layer. The buffer layer includes a predetermined flavor and ensures that the active layer does not interfere with the flavor of the inner layer. According to another embodiment, a cough suppressant system includes the cough suppressor and one or more auxiliary flavor tablets, each of which includes a desirable flavor such that this flavor may be selectively maintained long after the flavor of the inner layer is consumed. A cough suppressant liquid may be included for application to the flavor tablets.

### BACKGROUND OF THE INVENTION

This invention relates generally to cough suppressants and, more particularly, to a cough suppressor arranged in a flavorful gum drop configuration having both chewable and cough suppressant layers.

Flu and cold season causes many people to suffer from a persistent and nagging cough. Many cold remedies exist and are frequently consumed in order to remedy such a cough. Despite this availability, many people choose not to use these remedies due to their ineffectiveness or, more frequently, due to their generally unpleasant flavor.

Various products and proposals are known in the art for attempting to mask the otherwise undesirable test of cough suppressants and other medicine. For example, cough drops may be covered with a flavor layer in an attempt to mask the taste of the medicine layer. Although assumably effective for their intended purposes, the flavor and medicine layers become mixed together in a manner that eliminates the masking effect. After consuming the cough suppressant, the user is still left with an undesirable aftertaste in his mouth.

Therefore, it would be desirable to have a cough suppressor which effectively separates a medicine layer from a flavor layer and which leaves the consumer with a pleasant taste in his mouth after consuming the cough suppressor product. Further, it would be desirable to have a cough suppressor that includes chewing gum after the rest of the cough suppressor has been consumed.

### SUMMARY OF THE INVENTION

Therefore, a cough suppressor according to the present invention includes a chewable inner layer having a predetermined flavor. Preferably, the inner layer is chewing gum. The cough suppressor also includes an active layer situated outside the inner layer, the active inner layer including a cough suppressant. In addition, a buffer layer completely separates the inner layer from the active layer. The buffer layer includes a predetermined flavor and ensures that the active layer does not contact the inner layer.

The cough suppressor may include one or more flavor tablets which may or may not chewable. Each flavor tablet provides a person with an additional means for masking the aftertaste of the cough suppressant layer of the cough suppressor and for maintaining a desirable flavor. In addition, a liquid cough suppressant liquid may be included which may be sprayed or otherwise applied to each flavor tablet.

Therefore, a general object of this invention is to provide a cough suppressor having a cough suppressant for relieving a person's cough and that leaves a pleasant taste in the person's mouth.

Another object of this invention is to provide a cough suppressor, as aforesaid, that includes a flavor layer that masks the undesirable taste of a cough suppressant layer.

Still another object of this invention is to provide a cough suppressor, as aforesaid, that includes a flavored buffer layer that separates the cough suppressant layer from the flavor layer.

Yet another object of this invention is to provide a cough suppressor, as aforesaid, that includes a chewable inner layer.

A further object of this invention is to provide a cough suppressor, as aforesaid, in which a flavor of the cough suppressant (active) layer does not pass through the buffer layer to the flavor layer.

A still further object of this invention is to provide a cough suppressor, as aforesaid, that includes auxiliary flavor tablets enabling a person to maintain the desirable flavor after the flavor originally in the cough suppressor has been consumed.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a cough suppressor according to a preferred embodiment of the present invention;

FIG. 2a is a top view of the cough suppressor as in FIG. 1;

FIG. 2b is a sectional view taken along line 2b-2bof FIG. 2a;

FIG. 3 is a perspective view of a cough suppressor system according to another embodiment of the present invention; and

FIG. 4 is another perspective view of the cough suppressor system as in FIG. 3.

### DESCRIPTION OF THE PREFERRED EMBODIMENT

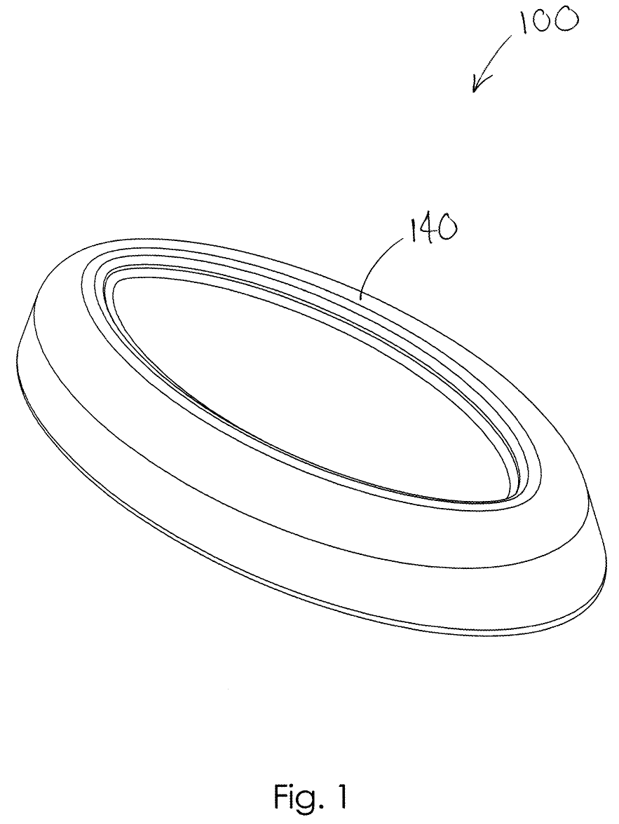

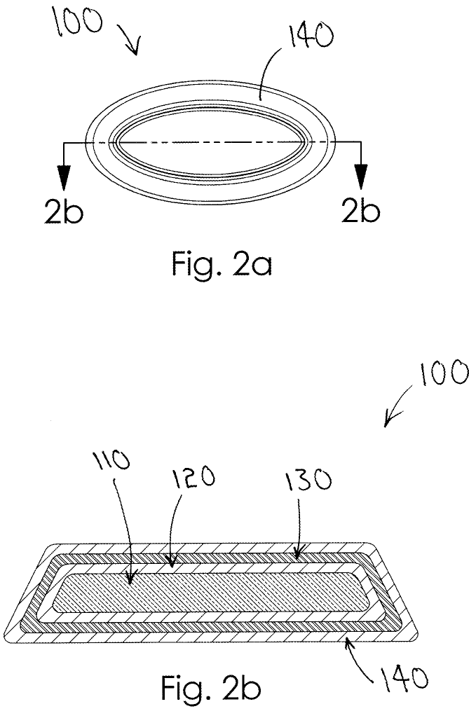

A cough suppressor 100 and a cough suppressing system 200 according to the present invention will now be described in detail with reference to FIGS. 1 through 4 of the accompanying drawings. More particularly, a cough suppressor 100 according to the current invention includes a plurality of layers having distinct properties, including a chewable inner layer 110, an intermediate layer (also referred to herein as a “buffer layer” or an “intermediate flavor layer”) 120, and an active layer 130 (FIG. 2b).

The chewable inner layer 110 has a predetermined flavor and may include chewing gum, taffy, or other long-lasting substances. The active layer 130 is outside the inner layer 110 and includes a cough suppressant, such as dextromethorphan, noscapine, ethyl morphine, codeine, menthol, zinc gluconate glycine, and/or pectin. The active layer 130 may have a solid or liquid consistency and a predetermined flavor which may include a masking agent. Though the predetermined flavor of the active layer 130 may generally be distinct from the predetermined flavor of the inner layer 110, this need not be required.

The intermediate layer 120 completely separates the inner layer 110 and the active layer 130, ensuring that the active layer 130 does not contact the inner layer 110. This separation allows the predetermined flavor of the inner layer 110 to remain untainted by the predetermined flavor of the active layer 130. The intermediate layer 120 may have a predetermined flavor that may be the same as or different from the predetermined flavor of the inner layer 110, and the intermediate layer 120 may be dissolvable in a human mouth.

An outer flavor layer 140 may surround the active layer 130, as shown in FIGS. 1 through 2b. The outer flavor layer 140 may have a predetermined flavor that is the same as or different from the predetermined flavor of the intermediate layer 120 or the inner layer 110, and the outer flavor layer 140 may be dissolvable in a human mouth.

In use, the cough suppressor 100 may be placed in a user's mouth. The outer flavor layer 140 may provide a pleasant flavor and dissolve to expose the active layer 130. The active layer 130 may be ingested through dissolving or by separating from the intermediate layer 120 and being swallowed, and the active layer 130 may prevent the user from coughing or otherwise benefit the user medically. Once the active layer 130 is ingested, the intermediate layer 120 is exposed to the user. The intermediate layer 120 may provide a pleasant flavor to overcome any unpleasant flavor of the active layer 130, and the intermediate layer 120 may dissolve to expose the inner layer 110 to the user. The user may chew the inner layer 110 to enjoy the predetermined flavor of the inner layer 110 and/or to overcome any lingering flavors of the active layer 120. It is notable that the inner layer 110 is not tainted by the active layer 120 because the predetermined flavor of the active layer 120 is not passable through the intermediate layer 120 to the inner layer 110.

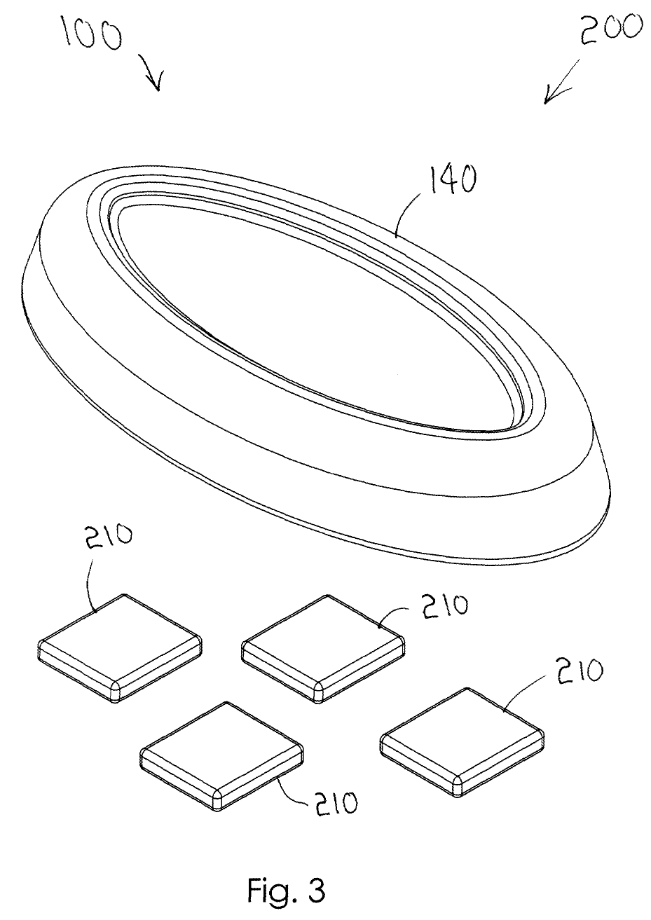

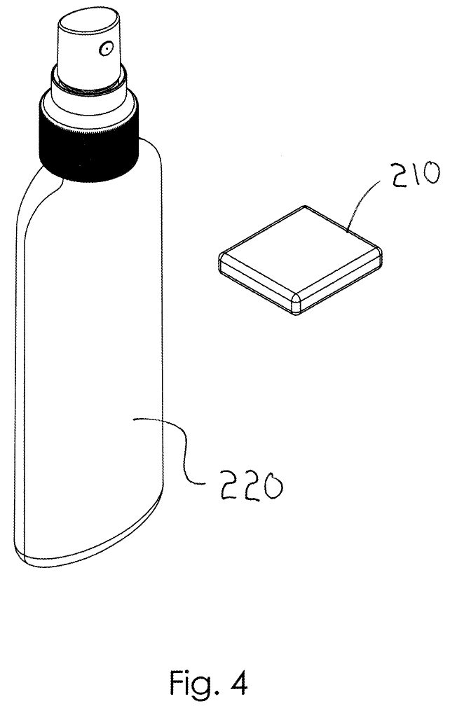

The cough suppressing system 200 according to the current invention includes a cough suppressor 100 as described above and one or more flavor tablets 210 separate from the cough suppressor 100 as shown in FIGS. 3 and 4. Each respective flavor tablet 210 may include chewing gum and may have a predetermined flavor. Preferably, the flavor tablet is chewable but one that dissolves in a person's mouth would also work. The predetermined flavor of the flavor tablet(s) 210 may or may not be the same as the predetermined flavor of the inner layer 110, the intermediate layer 120, or the outer layer 140. Thus, a pleasant taste may be selectively maintained in a person's mouth long after the flavor of the inner flavor layer 110 has been consumed.

As shown in FIG. 4, the cough suppressing system 200 may include a liquid cough suppressant 220 separate from the cough suppressor 100 and the flavor tablet(s) 210 for introduction onto the flavor tablet(s) 210.

In using the cough suppressing system 200, the cough suppressor 100 is used as described above. One or more flavor tablet 210 may then be placed in the user's mouth either in addition to the inner layer 110 or in replacement of the inner layer 110 after the inner layer 110 is utilized. If the user needs more cough suppressant, the liquid cough suppressant 220 may be introduced onto one or more tablet 210 (e.g., by spraying or dabbing), and the tablet 210 may be chewed or otherwise consumed by the user.

It is understood that while certain forms of this invention have been illustrated and described, it is not limited thereto except insofar as such limitations are included in the following claims and allowable functional equivalents thereof.

What is claimed is as follows:

1. A cough suppressor, comprising:
a chewable inner layer having a predetermined flavor;
an active layer outside said inner layer, said active layer including a cough suppressant; and
an intermediate flavor layer completely separating said inner layer and said active layer, said intermediate layer having a predetermined flavor.

2. The cough suppressor of claim 1, further comprising an outer flavor layer surrounding said active layer, said outer flavor layer having a predetermined flavor.

3. The cough suppressor of claim 2, wherein said outer flavor layer is dissolvable.

4. The cough suppressor of claim 3, wherein:
said chewable inner layer includes chewing gum; and
said active layer has a consistency selected from the group consisting of solid and liquid.

5. The cough suppressor of claim 1, wherein said cough suppressant is selected from the group consisting of dextromethorphan, noscapine, ethyl morphine, codeine, menthol, zinc gluconate glycine, and pectin.

6. The cough suppressor of claim 1, wherein said active layer has a liquid consistency.

7. The cough suppressor of claim 1, wherein said active layer has a solid consistency.

8. The cough suppressor of claim 1, wherein said chewable inner layer includes chewing gum.

9. A cough suppressor, comprising:
a chewable inner layer having a predetermined flavor;
an active layer situated outside said inner layer, said active layer including a cough suppressant; and
a buffer layer completely separating said inner layer and said active layer, said buffer layer having a predetermined flavor and ensuring that said active layer does not contact said inner layer.

10. The cough suppressor of claim 9, wherein said buffer layer is dissolvable in a human mouth.

11. The cough suppressor of claim 10, further comprising an outer flavor layer surrounding said active layer, said outer flavor layer having a predetermined flavor and being dissolvable in a human mouth.

12. The cough suppressor of claim 11, wherein:
said inner layer includes chewing gum; and
said active layer has a consistency selected from the group consisting of solid and liquid.

13. The cough suppressor of claim 12, wherein said cough suppressant is selected from the group consisting of dextromethorphan, noscapine, ethyl morphine, codeine, menthol, zinc gluconate glycine, and pectin.

14. The cough suppressor of claim 9, wherein said inner layer includes chewing gum.

15. The cough suppressor of claim 14, wherein:
said active layer includes a predetermined flavor;
said predetermined flavor of said active layer is distinct from said predetermined flavor of said inner layer; and
said predetermined flavor of said active layer is not passable through said buffer layer to said inner layer.

16. A cough suppressing system, comprising:
a cough suppressor comprising:
a chewable inner layer having a predetermined flavor;
an active layer outside said inner layer, said active layer including a cough suppressant; and
an intermediate flavor layer completely separating said inner layer and said active layer whereby said inner layer does not contact said active layer, said intermediate layer having a predetermined flavor;
a flavor tablet separate from said cough suppressor.

17. The cough suppressing system of claim 16, wherein:
said flavor tablet includes chewing gum; and
said flavor tablet has a predetermined flavor.

18. The cough suppressing system of claim 17, further comprising a liquid cough suppressant separate from said cough suppressor and said flavor tablet for introduction onto said flavor tablet.

19. The cough suppressing system of claim 18, wherein:
said cough suppressor includes an outer flavor layer surrounding said active layer, said outer flavor layer being dissolvable and having a predetermined flavor;
said chewable inner layer of said cough suppressor includes chewing gum; and
said active layer of said cough suppressor has a consistency selected from the group consisting of solid and liquid.

20. The cough suppressing system of claim 17, wherein:
said cough suppressor further comprises an outer flavor layer surrounding said active layer, said outer flavor layer being dissolvable and having a predetermined flavor;
said chewable inner layer of said cough suppressor includes chewing gum; and
said active layer of said cough suppressor has a consistency selected from the group consisting of solid and liquid.
